(19) United States
(12) Patent Application Publication
(10) Pub. No.: US 2014/0311214 A1
(43) Pub. Date: Oct. 23, 2014

(54) Portable Wet Calibration System for Handheld Breath Testers

(71) Applicant: Alcotek, Inc., St. Louis, MO (US)

(72) Inventor: Karl R. Wolf, JR., Eureka, MO (US)

(73) Assignee: Alcotek, Inc., St. Louis, MO (US)

(21) Appl. No.: 14/204,702

(22) Filed: Mar. 11, 2014

Related U.S. Application Data
(60) Provisional application No. 61/780,811, filed on Mar. 13, 2013.

(51) Int. Cl. G01N 33/00

(52) U.S. Cl. CPC G01N 33/0006
USPC 73/23.3

(57) ABSTRACT

Devices and systems for methods for calibrating breath alcohol testing and measuring equipment, or checking the accuracy of such equipment without calibration, using a permeable membrane to separate liquid and gas reservoirs and maintain an equilibrium state as samples are drawn from the device.

### CROSS REFERENCE TO RELATED APPLICATIONS

This application claims benefit of U.S. Provisional Patent Application No. 61/780,811 filed Mar. 13, 2013, the entire disclosure of which is incorporated herein by reference.

### BACKGROUND

1. Field of the Invention

This disclosure relates to the field of wet standard calibration systems and devices for use with handheld breath alcohol testing equipment that provide an accurate gas concentration for calibration or for checking accuracy without calibration.

2. Description of the Related Art

For the purposes of public safety on the roads and elsewhere, there is a need to make sure that individuals are not operating potentially dangerous machines (such as automobiles) while they are impaired by the effects of alcohol consumption. To try and prevent people from driving drunk, most states have enacted laws that impose fines or other criminal penalties if individuals have a breath or blood alcohol level above a certain amount. In order to effectively enforce these laws, it is necessary to be able to measure the alcohol concentration in human breath and compare the results against legal limits. There are a variety of measuring instruments used for determining the concentration of alcohol in human breath ranging from small hand held devices to larger bench top units and machines built into cars or certain machinery. Since a determination of breath alcohol above the legal threshold can result in criminal penalties, loss of a job, or other sanctions, the accuracy of these instruments is paramount.

Great care and effort is taken by owners and managers of evidential breath testing equipment to ensure proper calibration as well as follow-up accuracy checks at generally regular intervals. In attempts to eliminate the labor time of this testing and concerns about human error in the testing, manufacturers of breath testing equipment often offer automated or semi-automated methods for doing calibrations and accuracy checks. Some users of breath alcohol test equipment, such as Motor Vehicle Law Enforcement, may even require an automatic accuracy check every time they test a human subject and sometimes even before and after the human subject test simply to make sure that the device is reading correctly and will supply court-admissible evidence.

There are generally different standards used when calibrating breath testers. As breath (containing alcohol or not) is a vapor comprising exhalation gases and vaporized substances, instruments that measure alcohol concentration in this breath vapor generally need standards to be provided in a similar form for accurate calibration. Calibration gases of many sorts are well known in many applications including breath testing. In breath testing, the calibration standards are generally of two types, wet and dry. Wet standards include water vapor; dry standards do not. Some argue that wet standards are better because they include moisture like human breath and are therefore more representative. However, commercial providers of both wet and dry standards generally advertise +/−2% accuracy of calculations with actual breath.

In either case, the alcohol concentration of measurement interest is in a carrier gas such as air, breath, or nitrogen. A typical breath ethanol concentration which would result in illegal driving in most states is 200 parts per million (ppm) or more. That is 200 parts ethanol per million parts of carrier gas regardless of the carrier gas composition. Therefore, the standards generally provide samples that contain very close to 200 ppm to make sure the dividing line is correctly calibrated.

Wet standards have a long history in breath testing, are well accepted, and the liquids used in them can be certified by chemical analysis against NIST traceable standards. The standards are prepared by combining known amounts of ethanol and water in a partially filled jar that is accurately heated to 34° C. These heated jars are sold commercially and are referred to as Simulators. At equilibrium, the quiescent headspace above the jar contains a vapor with a known concentration of ethanol along with nearly 100% relative humidity at that temperature. In one special case of a wet standard, known as an “Equilibrator,” no heating is used, but the operator is required to read its temperature (usually equal to ambient) and follow a lookup table to see what gas concentration is delivered when similarly blown through as in a standard simulator.

By introducing sober human breath or air from another suitable source into the jar (by blowing or injecting gas into the liquid), the known concentration of ethanol vapor exits the headspace and can be introduced into a breath tester at which point a measurement may be taken. Typically, a liter or more of gas is blown through the simulator for each test. As newly introduced air or breath bubbles up through the liquid, it replaces the gas exiting the simulator with newly equilibrated gas.

Generally, the simulators of the prior art go to great lengths to keep the temperature of the system constant at 34° C. +/−0.1° C. This is because, as the temperature changes, so does the equilibrium point. Thus, the alcohol concentration in the gas varies with the temperature of the system. For example, at 34° C., a 0.1° change can represent well over a ½% change in the gas. Notably, this air/water equilibrium relationship for ethanol over temperature is not linear. Those skilled in the art will recognize, as demonstrated in the partition data below (from R. N. Harger, et al., 1949) that the ratio of ethanol concentration in the air to the water goes up in a non-linear fashion as the temperature goes up:

Harger Data

| ° C. | K_{A/W} × 10³ |
|---|---|
| 1 | 0.035 |
| 5 | 0.046 |
| 10 | 0.073 |
| 15 | 0.107 |
| 20 | 0.155 |
| 25 | 0.217 |
| 30 | 0.310 |
| 35 | 0.418 |
| 37 | 0.470 |
| 40 | 0.562 |

Dry standards, by contrast, have no water vapor included with them. This is because dry standards are prepared with carrier gases such as nitrogen or argon and are supplied in pressurized tanks ranging from 500-2500 psi. At these pressures, if water vapor were included in amounts similar to human breath concentrations in practical field use, the water would condense out of the gas, trap ethanol, and cause wholly inaccurate results. The dry gas standards are typically certified by measurement against NIST-prepared standards.

In automated wet testing, the above-mentioned Simulators generally have input and output ports. Typically, a Simulator will sit alongside a breath test machine, normally on a desktop. The output of the Simulator is plumbed into the instrument such that, when gas is pumped into the Simulator input (either from a tester blowing into it or from an associated gas tank or pump), a vapor of known ethanol concentration will be presented for measurement or calibration in the same manner human breath would be. Typically, an electric pump is used to pump ambient air into the Simulator for this purpose. The pump may be internal to the breath tester, part of the Simulator itself, or an entirely separate component. Typically, gas is pumped through a Simulator for four to ten (4-10) seconds in order for a measurement to be completed. This pump time varies depending on the flow rate and the amount of instrument volume that has to be purged of ambient gas before a measurement is taken to ensure the measurement is taken of the carrier gas with the correct concentration of ethanol. It is not unusual in these cases that about one liter of air is pumped through the simulator with every test.

Every time a sample is taken from a Simulator, some of the ethanol in the liquid replenishes lost ethanol from the headspace. Thus, over time, the equilibrium concentration of ethanol provided by the Simulator decreases from its originally intended value as ethanol is slowly lost to the ambient air due to the carrier gas (and the carried ethanol) being exhausted from the breath tester. Some breath test instruments use recirculation systems that take the ethanol vapor provided by the Simulator output, after it exits the breath tester's measurement chamber or manifold and pumps it back into the Simulator inlet, instead of using ambient air to provide the simulated exhalation. This greatly reduces any effects of lost ethanol from the Simulator causing lower concentrations to be provided over time, since used ethanol is not exhausted to the ambient but is returned to the Simulator.

Whether using recirculation systems or not, care must be taken to avoid any condensation of water from the Simulator output until the concentration of ethanol is measured by the breath tester. Otherwise, the alcohol in the gas will be less than intended due to ethanol being condensed from the gas. To avoid condensation, various elements or tubes in the instrument are generally heated prior to measurement.

It should be noted that using Simulators for portable instruments or in on-site calibration tests can be problematic. They are subject to splashing, tipping over, and operate properly only within a very limited ambient temperature range due to their complicated design which is necessary for accuracy. Further, they are not really designed for easy or efficient transport and that tends to limit their use to controlled settings.

The dry gas standards are provided in a variety of types of high-pressure cylinders. A typical size of a tank is approximately one (1) liter or more. These cylinders are typically equipped with pressure regulators where the high tank pressure is regulated down to a much lower delivery pressure to the breath tester to better simulate the pressure provided by human breath. Often, an electronic shut-off valve will allow delivery of the low-pressure calibration gas to the measurement chamber on demand.

Compared to wet standards, dry standards offer some advantages. Dry gas delivery systems generally represent a less complex hardware system design to provide automated calibrations and accuracy checks than the wet standards. The dry gas system is generally easier for instrument owners to manage and maintain and the dry gas system is certainly more amenable to a portable system. Specifically, since the only major components of a dry gas system are the tank and regulator, they are pretty easily portable and are not as affected by movement or situation as wet systems. The dry gas tanks will eventually run empty, but no recirculation system is required to keep the value stable throughout the tank's lifetime.

However, dry gas standards have several factors that complicate their use. First of all, they require a compensation for barometric pressure in the breath tester. The concentration of dry gas standards follow the ideal gas law, and the measured value will change with barometric pressure changes due to elevation or weather. Also, if a dry gas system has a leak, it is possible to lose a significant amount of gas before a problem is noticed. Furthermore, some users (especially mobile ones) have concerns about the safety of transporting even relatively small high-pressure gas tanks that, even while filled with generally nonflammable gas, are potentially explosive due to their high pressure. Lastly, as stated earlier, the dry gas contains no water vapor. Some individuals skilled in the art believe that a water component to the calibration gas is essential, because water vapor is a large constituent of human breath and it would therefore be possible to challenge the reading of a breath tester which has only been calibrated using a dry gas system.

### SUMMARY

The following is a summary of the invention which should provide to the reader a basic understanding of some aspects of the invention. This summary is not intended to identify critical components of the invention, nor in any way to delineate the scope of the invention. The sole purpose of this summary is to present in simplified language some aspects of the invention as a prelude to the more detailed description presented below.

Because of these and other problems in the art, described herein, among other things, is a wet standard calibration device, the device comprising: a housing, the housing defining a liquid reservoir and a gas reservoir; a gas permeable membrane separating the liquid reservoir and the gas reservoir; wherein a simulator solution comprised water and ethanol is located in the liquid reservoir; wherein a gas solution comprised of water vapor and alcohol vapor is located in the gas reservoir; wherein the alcohol concentration of the simulator solution is at an equilibrium state with the alcohol concentration of the gas solution; and wherein, as any gas leaves the gas reservoir, the lost alcohol molecules are replenished from the liquid simulator solution, returning the equilibrium state.

In an embodiment, the device is further comprised of a septum, the septum being located in the housing of the gas reservoir.

In another embodiment, the device is further comprised of a means for replenishing the simulator solution.

In another embodiment, the housing is comprised of a material with a large thermal mass.

In a further embodiment, the material with the large thermal mass is brass.

In another embodiment, the device further comprises an insulative means, which insulative means is attached to the housing.

In another embodiment, the housing is comprised of a material with anti-microbial properties.

In a further embodiment, the housing material is chosen from the group consisting of: copper and brass.

In another embodiment, the device is further comprised of a temperature sensor affixed to the housing.

In another embodiment, the device is further comprised of a computer assembly with a user interface.

In another embodiment, the device is further comprised of a heating mechanism.

### BRIEF DESCRIPTION OF THE DRAWINGS

FIG. 1 shows an embodiment of a portable wet calibration device.

FIG. 2 shows an embodiment of a portable wet calibration device in use.

FIG. 3 shows an embodiment of a portable wet calibration device in various orientations.

FIG. 4 provides an embodiment of a box or bag of solution keyed to the liquid fill port.

FIG. 5 provides an embodiment of a filling system where a bag or box is burst in situ as a cover is sealed.

FIG. 6 provides an embodiment of a pre-filled liquid reservoir that is a separate component attached to the gas reservoir at the time of use.

FIG. 7 shows how an embodiment of the intake port of a breath alcohol tester can be connected to an intake straw.

### DESCRIPTION OF THE PREFERRED EMBODIMENT(S)

Described herein are wet standard calibration systems and devices for use with handheld equipment that provide an accurate gas concentration for calibration or for checking accuracy without calibration.

As mentioned above, automated dry and wet gas systems are typically used with stationary and, in some cases, transportable instruments sitting on a benchtop. These same standards (typically in a non-automated fashion) are used for calibrating and checking the accuracy of small, portable handheld breath testers as well. In the case of a handheld instrument, typically far less gas is required per test than in desktop systems. It is in this arena of handheld testers where the system described herein is focused. In particular, embodiments of the invention described herein fill a need in the art for a small wet standard that is extremely portable and provides an accurate gas concentration for calibration—i.e., an ideal simulator for handheld breath test instruments. Among other things, the portable wet simulator described herein is a long-lasting, no-spill device, requires no special shipping due to having no pressurized gas, and minimizes any depletion issues. The device also uses the same wet standard solutions that are commonly available for existing wet simulators, albeit in much smaller quantities. Finally, the device delivers calibration gas at an extremely low cost per unit.

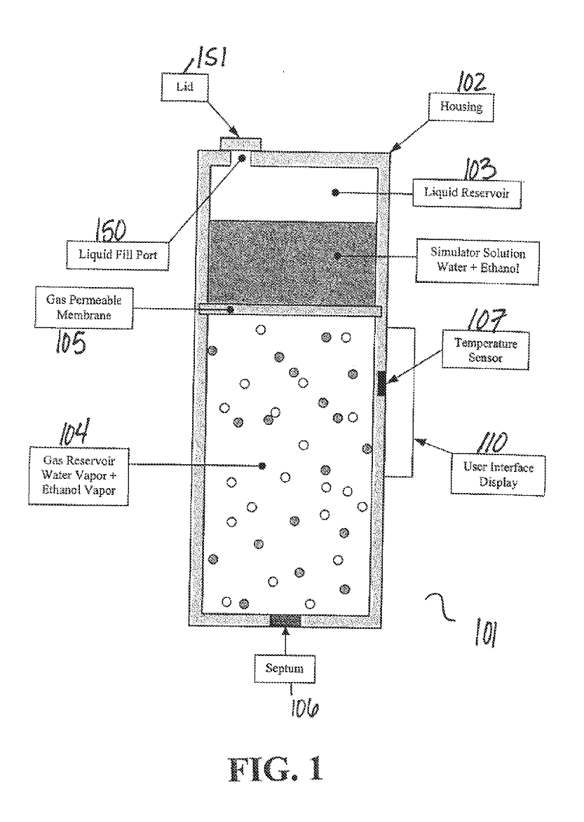

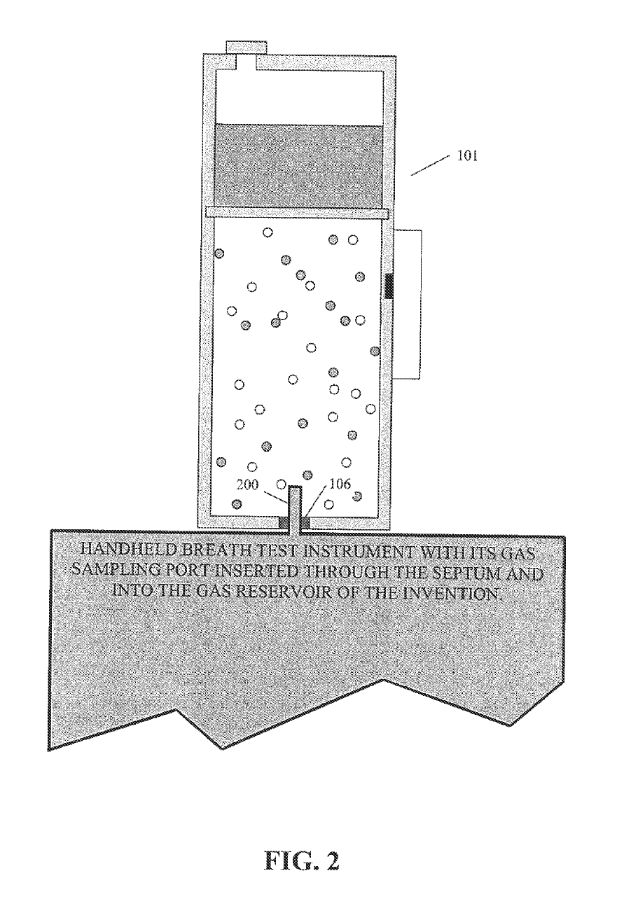

In embodiments, as demonstrated in FIGS. 1 and 2, the wet standard calibration device/simulator (101) consists of a housing (102) which contains two volumes defined by a liquid reservoir (103) and a gas reservoir (104) which are, in turn, separated by a gas permeable membrane (105). In certain embodiments, as depicted in FIGS. 1 and 2, the gas reservoir (104) is larger in capacity than the liquid reservoir (103), but this proportion is not necessary. With regard to the gas permeable membrane (105), it should be understood that any gas permeable membrane known to those of ordinary skill in the art through which alcohol vapors can travel to reach equilibrium is contemplated in this disclosure.

In the embodiments, depicted in FIGS. 1 and 2, the gas reservoir (104) portion of the housing (102) contains a septum (106) in its wall. The position of the septum (106) is not determinative, it may be located anywhere in the housing (102) of the gas reservoir (104). The septum (106) functions to keep gas from leaking out to any appreciable extent when the simulator (101) is not in active use. When the simulator (101) is in active use, as demonstrated in FIG. 2, the gas sampling port (200) of a handheld breath test instrument is inserted through the septum (106) to take a gas sample as required in a manner understood by those of ordinary skill in the art. Generally, when the gas sampling port is inserted through the septum (106), no gas leaks out of the simulator (101) to any appreciable extent.

It should be understood that, when the simulator (101) is in active use, the simulator (101) replaces the intake straw (201) component of the prior art handheld breath test instruments (i.e., the component into which an individual breathes when performing the test). Thus, the gas solution inside the gas reservoir (104) of the simulator (101), when in use with the gas sampling port (200) of a handheld breath test instrument inserted through the septum (106), simulates the internal space of the intake straw (201).

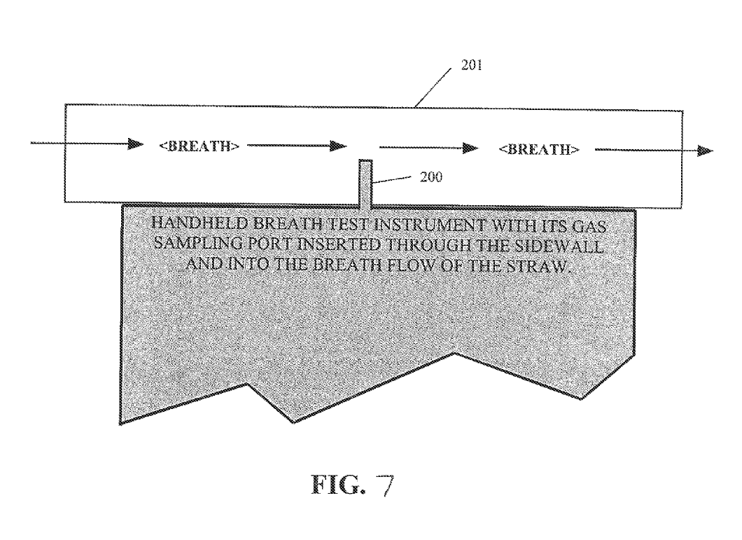

Accordingly, the simulator will only be placed on the sampling port (200) when the handheld breath test instrument is not in use for an individual breath test, and only for a time long enough to run a calibration or accuracy check on the instrument. Clearly, prior to any individual test being performed, the simulator (101) must be removed and replaced with the intake straw (201) as shown in FIG. 7. After an individual test is completed, the intake straw (201) could be removed and the intake straw (201) will again be replaced with the simulator (101) to recalibrate or check the accuracy of the handheld breath instrument.

Further, in another embodiment, as depicted in FIGS. 1 and 2, the liquid reservoir (103) portion of the housing (102) contains a means for adding simulator solution to the liquid reservoir (103). In the embodiment depicted in FIGS. 1 and 2, this means comprises a liquid fill port (150) and a lid (151) to the liquid fill port (150). In this embodiment, the liquid fill port (150) comprises an opening in the housing (102) that comprises the liquid reservoir (103). When not in use, the liquid fill port (150) is covered with the lid (151). To add more simulator solution to the liquid reservoir (103), the lid (151) is removed and simulator solution is poured into the liquid reservoir (103) via the liquid fill port (150).

In another embodiment, it is contemplated that vents may be required in either the gas or liquid reservoirs. Such vents could additionally contain check valves allowing venting in only one direction and to prevent leaks otherwise.

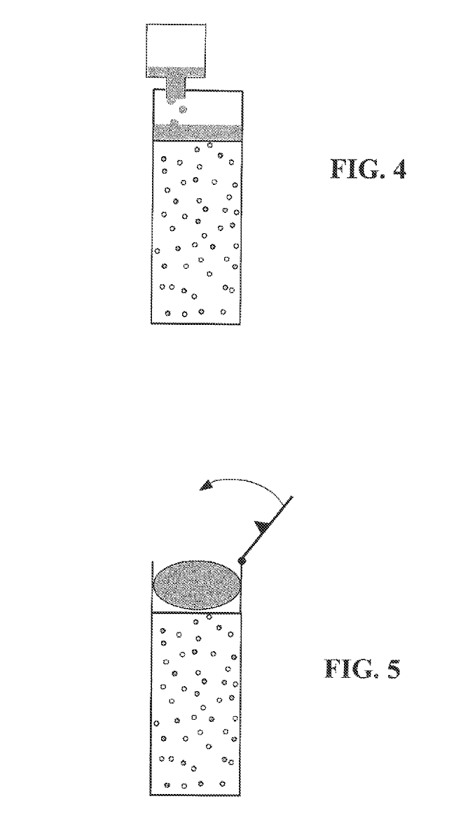

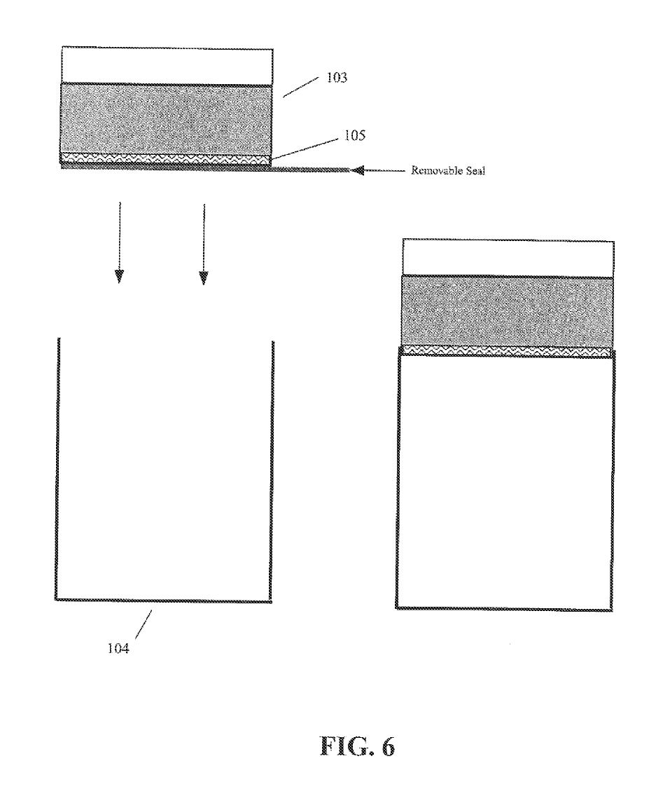

Alternatively, other embodiments are contemplated. A box or bag of solution pre- measured to the correct fill amount could be supplied and could be keyed to the liquid fill port for simple, no-spill filling as in FIG. 4. An entire bag of pre-measured liquid could be inserted into the liquid reservoir and made to burst in situ as a cover is sealed over it as in FIG. 5. The entire pre-filled liquid reservoir (103) could be a separate component that could be attached to the gas reservoir (104) at the time of use and activated before attachment by removing a seal as in FIG. 6. Such an attachable liquid reservoir could or could not contain the gas permeable membrane (105).

Since temperature plays an important role in how the gas and liquid partition in such a device (101), and thus what the exact concentration of ethanol is in the gas reservoir, in certain embodiments, it is contemplated that the housing material of the wet standard calibration device (101) will be a material with a high thermal mass such as brass in order to keep the temperature of the entire device uniform. However, this is not limiting and any other material known to those of ordinary skill in the art for creating housing including, but not limited to, plastics, metals and woods, is contemplated in this application. Further, because it is often desirable to force any change in temperature of the device (101) due to ambient temperature changes to be slow moving, in certain embodiments an insulation means known to those of ordinary skill in the art is added to the device (101) outside of the housing (102) of high thermal mass. In these embodiments, the insulation would add stability to the simulator (101). In certain embodiments, the housing (102) will be comprised of insulation outside of a material wall with a low thermal mass. In addition to the thermal mass, the inhibition of bacterial growth is another selection criteria for the housing (102) material of the device (101). Accordingly, in certain embodiments, materials such as copper and brass that have natural anti-microbial properties and certain formulations of thermoplastics specifically engineered for anti-microbial properties are contemplated as materials for the housing (102). Anti-microbial coatings known to those of ordinary skill in the art that are applied to the housing (102) are also contemplated.

In order to assist in the regulation of temperature in the device (101), in certain embodiments, as depicted in FIGS. 1 and 2, a precision temperature sensor (107) is mounted against, or embedded in, the housing (102). The location of the precision temperature sensor (107) is not determinative as long as it is able to represent a temperature of the device (101) to at least +/−0.1° C. accuracy. In alternative embodiments, the precision temperature sensor (107) could be mounted such that it is directly exposed to the liquid or gas inside the housing (102).

In certain other embodiments, it is contemplated that the simulator (101) will have an electronic/computer assembly with a display that will serve as a user interface (110). In this embodiment, the electronic/computer assembly would know the nominal value of the liquid being used, take into account the temperature of the device (101), and report a gas concentration value in familiar units of measure for the operator to use when calibrating or checking the accuracy of a particular handheld instrument. In other embodiments, the user interface (110) will simply report the temperature and the calculations will be performed externally. In certain other embodiments, the user interface (110) would further include a means of changing the units of measure and using a variety of nominal values of the liquid. It is also contemplated that, in certain embodiments, the electronic/computer assembly which serves as the user interface (110) will include a counter so that the operator knows how many tests have been taken from the simulator (101) as a means to know when to replenish the simulator solution. Similarly, it is contemplated that, in some embodiments, the electronic/computer assembly will also include a time counter as a means to know when the shelf-life of the simulator solution has expired during use.

In still another embodiment, it is contemplated that the simulator (101) will also include a heating mechanism (120) known to those of ordinary skill in the art. In these embodiments, the heating mechanism (120) functions to maintain a specific temperature and partition ratio or a minimum temperature and partition ratio. Generally, any means for heating or maintaining a specific temperature or temperature range known to those of ordinary skill in the art are contemplated as potential heating mechanisms. Heating could be used in combination with insulation as earlier described.

In general, the wet standard calibration device (101) described herein functions in the following manner. As depicted in FIGS. 1 and 2, a simulator solution comprised of water and ethanol, in ratios known to those of ordinary skill in the art, is located in the internal chamber of the liquid reservoir (103). Further, a gas solution comprised of a combination of water vapor and ethanol vapor, in ratios known to those of ordinary skill in the art, is located in the gas reservoir (104). At a given temperature known to those of ordinary skill in the art, the alcohol concentration of the liquid reaches an equilibrium state with the alcohol concentration of the gas. As any gas that leaves the gas reservoir (104) (e.g., by a small leak or by sampling with an instrument), that gas (lost water vapor and ethanol vapor) is replenished from the liquid in the liquid reservoir (103) by travelling through the gas permeable membrane. This transport of molecules from the simulator solution to the gas solution returns the gas solution equilibrium concentration for that temperature. Because the liquid simulator solution has thousands of times more ethanol molecules than the gas solution and because the typical handheld breath tester will only remove a very small portion of the volume of the gas solution for a given test, the simulator solution can keep the gas concentration of the gas solution in the gas reservoir (104) constant (i.e., at the equilibrium concentration) for a very long period of time at a given temperature or keep the gas concentration accurate compared to the concentration reported in the user interface for a very long time at different and changing temperatures.

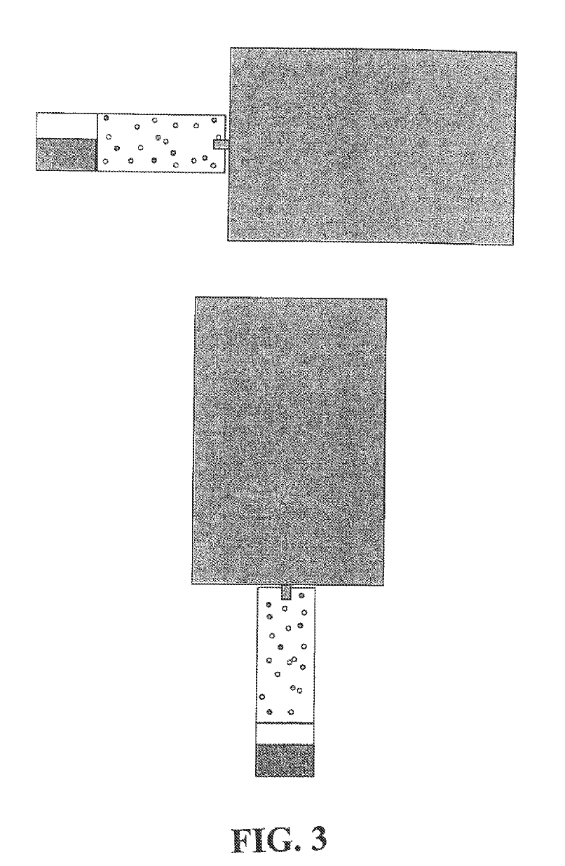

Notably, as demonstrated in FIG. 3, it should be understood that the simulator (101) can be used in any orientation—e.g., vertical or horizont—without affecting its operation.

It should be understood that, as well as being a simulator that is capable of delivering known gas concentrations to handheld breath test instruments for their calibration and/or accuracy checks, the simulator could be used with a handheld breath test instrument that is already calibrated to identify an unknown concentration of a fairly small liquid sample.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

1. A wet standard calibration device, the device comprising:
a housing, the housing defining a liquid reservoir and a gas reservoir;
a gas permeable membrane separating the liquid reservoir and the gas reservoir;
wherein a simulator solution comprised water and ethanol is located in the liquid reservoir;
wherein a gas solution comprised of water vapor and alcohol vapor is located in the gas reservoir;
wherein the alcohol concentration of the simulator solution is at an equilibrium state with the alcohol concentration of the gas solution; and
wherein, as any gas leaves the gas reservoir, the lost alcohol molecules are replenished from the liquid simulator solution, returning the equilibrium state.

2. The device of claim 1, the device being further comprised of a septum, the septum being located in the housing of the gas reservoir.

3. The device of claim 1, further comprising a means for replenishing the simulator solution.

4. The device of claim 1, wherein the housing is comprised of a material with a large thermal mass.

5. The device of claim 4, wherein the material with the large thermal mass is brass.

6. The device of claim 1, further comprising an insulative means, the insulative means being attached to the housing.

7. The device of claim 1, wherein the housing is comprised of a material with anti-microbial properties.

8. The device of claim 7, wherein the material is chosen from the group consisting of:
copper and brass.

9. The device of claim 1, the device further comprising a temperature sensor affixed to the housing.

10. The device of claim 1, the device further comprising a computer assembly with a user interface.

11. The device of claim 1, the device further comprising a heating mechanism.
